(19) United States
(12) Patent Application Publication
(10) Pub. No.: US 2024/0203596 A1
(43) Pub. Date: Jun. 20, 2024

(54) SYSTEMS AND METHODS FOR PREDICTING A CONDITION

(71) Applicant: Lindsay Lifescience LLC, Moab, UT (US)

(72) Inventors: Marc Lindsay, Moab, UT (US); Dave Bierschied, Moab, UT (US); Hugh Napier, Gauteng (ZA)

(73) Assignee: Lindsay Lifescience LLC, Moab, UT (US)

(21) Appl. No.: 18/554,505

(22) PCT Filed: Apr. 11, 2022
(86) PCT No.: PCT/US22/24308

§ 371 Date: Oct. 9, 2023

Related U.S. Application Data
(60) Provisional application No. 63/172,838, filed on Apr. 9, 2021.

(51) Int. Cl. G16H 50/20; A61B 5/00; A61M 37/00; G16H 20/00

(52) U.S. Cl. CPC G16H 50/20; A61B 5/6833; A61B 5/7267; A61B 5/7405; A61B 5/742; A61M 37/0015; G16H 20/00; A61M 2037/0046; A61M 2037/0061

(57) ABSTRACT

A system and method for predicting a condition is described herein. A patch that affixes to a subject communicates with a computing device to allow one to predict a condition before it happens, during the occurrence of the condition, or after the occurrence of the condition.

### CROSS-REFERENCE TO RELATED APPLICATIONS

This application claims the benefit of U.S. Provisional Application No. 63/172,838 filed on Apr. 9, 2021, which is incorporated herein by reference in their entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### FIELD

The present teachings relate to systems and methods for predicting a condition using a patch affixed to a subject.

### INTRODUCTION

An aim of modern medicine is to provide personalized or individualized treatment regimens. Those are treatment regimens that take into account a patient's individual needs or risks. A particularly important risk is the presence of a cardiovascular complication, particularly an unrecognized cardiovascular complication, or a predilection for such cardiovascular complications. Cardiovascular complications, particularly heart diseases, are the leading cause of morbidity and mortality. Cardiovascular complications are not always readily diagnosed or detected easily and underlying conditions can remain asymptomatic for long periods of time. Therefore, reliable differential diagnosis of the presence of a cardiovascular complication is more difficult and error prone than generally believed. Specifically, patients suffering from symptoms of an acute cardiovascular event (e.g., myocardial infarction or MI) such as chest pain are currently subjected to an assay of cardiac troponin levels. To this end, cardiac troponin levels of the patients need to be determined. Indeed, the presence or lack of biomarkers, which include proteins, antibodies, cells, small chemicals, hormones and nucleic acids, may indicate a disease or a condition, and such biomarkers are oftentimes found in biological fluids within the body. For instance, blood serum and their levels are routinely measured for research and for clinical diagnosis. Standard tests include antibody analysis for detecting infections, allergic responses, and blood-borne markers. Blood is not the only biological fluid in which biomarkers reside, though, making it unsuitable for detecting some conditions, such as conditions that originate in solid tissues, and whilst this problem has been partially solved by taking tissue biopsies, such a process is time-consuming, painful, risky, costly. Interstitial fluid, extracellular fluid, plasma, and cerebrospinal fluid are all biological fluids that may also carry biomarkers of interest for detecting diseases or conditions.

Determining the likelihood of a condition is rather difficult, as symptoms oftentimes are ignored or only realized at the onset of the condition. A means for predicting whether a condition is upcoming based on continuously monitoring a person would be beneficial. In addition, being able to accumulate data to be able to better predict future conditions would be quite valuable.

### SUMMARY

The present teachings include a system for detecting a condition, comprising a patch, which is further comprised by at least one hollow microneedle, an assay compartment that prevents backflow into a subject comprising at least one reagent chamber, at least one detection chamber and at least one waste chamber; an adhesive that affixes the patch to a subject, a microprocessor that controls collection rate of a fluid, collects assay data, and communicates the assay data to a computing device, circuitry that creates an audio tone, and a cap that ensures the integrity of the system. The computing device is in communication with the patch, and optionally comprises at least one neural network that trains the system to better predict the condition. The computing device has memory, a processor, and a server, and is capable of processing data from the patch. The microneedle may be a single needle. In another embodiment, there is an array of microneedles. At least one reagent of the assay compartment may be mixed with the fluid drawn by the microneedles; this mixture ultimately is used to detect a biomarker or biomarkers of interest. In an embodiment, cardiac troponins (C, I and T) are the biomarkers of interest. In another embodiment, a ceramide is the biomarker of interest. Ceramides may include Cer(18:1/18:0), Cer(18:1/16:0), Cer(18:1/24:1), Cer(18:1/18:0)/24:0, Cer(18:1/16:0)/24:0, Cer(18:1/24:1)/24:0, Cer(d18:1/14:0), Cer(d18:1/16:0), Cer(d18:1/18:0), Cer(d18:1/18:1[9Z]), Cer(d18:1/20:0), Cer(d18:1/20:4), Cer(d18:1/22:0), Cer(d18:1/22:1), Cer(d18:1/23:0), Cer(d18:1/24:0), Cer(d18:1/24:1[15Z]), Cer(d18:1/25:0), Cer(d18:1/26:0), Cer(d18:1/26:1[17Z]), DHCer(d18:0/16:0), DHCer(d18:0/18:0), DHCer(d18:0/20:0), DHCer(d18:0/22:0), DHCer(d18:0/24:0), DHCer(d18:0/24:1[15Z]), and combinations thereof. In one embodiment, a multiplex reaction is envisioned where more than one biomarker is tested at the same time. The multiplex reaction could include biomarkers for one or more conditions and would include independent biomarkers to confirm fidelity of the assay. In some embodiments, cardiac troponins, nourin or ceramides are biomarkers of interest. The assay compartment may have at least one detection chamber where one or more reagents from respective reagent chambers is mixed with the fluid. The assay compartment may also have at least one waste chamber, which may accept waste product. The adhesive that affixes to a subject must be non-toxic, hypoallergenic, and be able to adhere to a subject for at least 12 hours and not elicit an allergic reaction. The adhesive also minimizes immune response, namely the production of mast cells. The patch includes a base substrate with at least one hollow microneedle. Microneedles may be affixed to a backing layer by an adhesive layer disposed between the backing layer and the back side of the base substrate. In some embodiments, the backing layer may include a tab portion that extends away from the microneedles. Alternatively, the tab portion may be disposed in a separate layer. Thus, the tab portion may be in the same plane or a different plane than the backing layer. The backing layer may be a composite material or multilayer material including materials with various properties to provide the desired properties and functions. For example, the backing material may be flexible, semi-rigid, or rigid, depending on the particular application. As another example, the backing layer may be substantially impermeable, protecting the one or more microneedles (or other components) from moisture, gases, and contaminants. Various polymers, elastomers, foams, paper-based materials, foil-based materials, metallized films, and non-woven and woven materials may be used as the adhesive. The backing layer may be temporarily or permanently affixed to a base substrate by the adhesive layer. In some embodiments, the adhesive layer may be disposed primarily in the body portion of the patch between the base substrate and backing layer. For example, the adhesive layer may be disposed between the base substrate and backing layer, and may extend beyond the base substrate to help adhere the patch to the patient's skin during application. The portion of the adhesive layer extending beyond the base substrate also may function to adhere the patch to a tray or container covering the microneedles during shipping and storage, as well as for disposal after its use. In an embodiment, the circuitry may be incorporated into the microprocessor. In another embodiment, the circuitry may be separate from the microprocessor. The audio tone may be for many purposes, one of which is the start of drawing of fluid. The audio tone may also be for the ending of drawing of fluid. The audio tone may also indicate reaching a particular level of a biomarker. The audio tone may also indicate the transfer of data from the patch to the computing device. The computing device may take on many forms. In an embodiment, the computing device is a smartphone. In another embodiment, the computing device is a computer. In another embodiment, the computing device is a smartwatch. At least one neural network may be used to continuously train the system. As biomarker data is collected, the system may get better at predicting the onset of a condition. The circuitry may comprise at least one electrode operably connected to the circuitry and at least one sensor operably connected to at least one electrode. In an embodiment, the patch is battery powered. In another embodiment, the patch is battery powered and rechargeable. The system may predict several conditions, one of which is acute myocardial infarction. In other embodiments, the system may predict infectious diseases, biological poisons, radiation, tropical diseases, and COVID-19. In other embodiments, the system may be used to determine an action based on biomarker information. For instance, at a certain biomarker level, the system may indicate that it is necessary to go to a healthcare facility. In another instance, the system may indicate that no action is required, or to self monitor.

In accordance with a further aspect, the at least one hollow microneedle draws fluid from the subject to monitor the presence of at least one biomarker by way of at least one of an osmotic gradient and a pumping mechanism. As the concentration of the biomarker initially is greater in the subject than in the microneedles, this difference in concentration will cause fluid to go from the subject to the microneedle. A pumping mechanism may speed up the process of drawing fluid into the microneedle and allow for predictable sampling intervals.

In accordance with yet another aspect, the at least one hollow microneedle draws fluid from a subject at least one of continuously and intermittently. When drawn intermittently, the fluid is drawn at a frequency based on the condition being assessed and clinical need. Patients more inclined to have a condition are tested more frequently, while those who show no obvious symptoms are tested less frequently.

In accordance with yet a further aspect, data associated with the fluid is transferred from the microprocessor to the computing device. Data transferred from the microprocessor to the computing is in regards to at least one biomarker.

In accordance with yet another aspect, at least one factor is used to predict the onset and progression of a condition by mapping the time to peak levels of one or more biomarkers.

In accordance with yet another aspect, knowledge of the at least one factor is used to recommend a course of action. A potential course of action is going to a hospital or healthcare facility. Once time to peak of biomarker levels is known, it is possible to offer additional courses of action.

In accordance with yet another aspect, the at least one neural network accepts the data, and a collection of output data of the at least one neural network is used to predict the onset of the condition. Continual refinement of the neural network as use of the system increases allows the system to better predict the onset of the condition.

In accordance with yet another aspect, the at least one hollow microneedle gains access to the fluid by at least one of percutaneously, dermally, sub-dermally, intra-peritoneally, and peritoneally.

In accordance with yet another aspect, the at least one biomarker is detected at least one of before the condition, during the condition, and after the condition.

The present teaching also include a method for predicting a condition in a subject. The method comprises providing a system for detecting a condition in a subject, with the system comprising a patch, further comprising: at least one hollow microneedle, an assay compartment that prevents backflow into a subject comprising at least one reagent chamber, at least one detection chamber and at least one waste chamber; an adhesive that affixes the patch to a subject, a microprocessor that controls collection rate of a fluid, collects assay data, and communicates the assay data to a computing device, circuitry that creates an audio tone, and a cap that ensure integrity of the system; a computing device in communication with the patch; and optionally at least one neural network that trains the system to better predict the condition; affixing the patch to a subject so that the at least one hollow microneedle accesses a fluid; drawing the fluid through the at least one hollow microneedle; detecting a level of at least one of a protein and a biomarker; monitoring the level of at least one biomarker; and predicting an onset and or progression of a condition based on the level of at least one biomarker.

In accordance with a further aspect, the audio tone may serve multiple functions, including alerts relating to the initiation and completion of an assay, results of the assay being sent to a smart device or computer as well as a warning tone indicating the onset of a condition.

In accordance with yet another aspect, at least one hollow microneedle draws fluid from the subject to detect the presence of at least one biomarker by way of at least one of an osmotic gradient and/or a pumping mechanism.

In accordance with yet another aspect, at least one hollow microneedles draws fluid from a subject at least one of continuously and intermittently.

In accordance with yet another aspect, data associated with the assay is transferred from the microprocessor to the computing device.

In accordance with yet another aspect, at least one factor is used to predict an onset of the condition, with the at least one factor being at least one of time to peak of biomarker levels and time to peak of biomarker levels.

In accordance with yet another aspect, knowledge of the at least one factor is used to recommend a course of action such as admitting oneself to hospital, or consulting a GP.

In accordance with yet another aspect, at least one neural network accepts the data, and a collection of output data of at least one neural network is used to predict the condition.

In accordance with yet another aspect, at least one hollow microneedle gains access to the fluid by at least one of subcutaneously, percutaneously, dermally, sub-dermally, intra-peritoneally, and peritoneally.

In accordance with yet another aspect, at least one biomarker is detected at least one of before the condition, during the condition, and after the condition.

In accordance with yet another aspect, a threshold associated with the at least one biomarker determines whether treatment is necessary.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1. Depiction of the patch.

FIG. 2. Another depiction of the patch.

FIG. 3. Depiction of a subject using the system.

FIG. 4. Flowchart for using the system.

FIG. 5. Flowchart of training process.

FIG. 6. Flowchart of assay compartment steps.

FIG. 7. Exemplary depiction of the assay compartment.

FIG. 8. Exemplary depiction of information flow between the layers of a neural network

FIG. 9. Exemplary depiction of a neural network with an input layer, a hidden layer, and an output layer.

FIG. 10. The steps for training the neural network.

FIG. 11. A functional block diagram showing the interaction between the microprocessor and the computing device.

### DETAILED DESCRIPTION

### Abbreviations and Definitions

To facilitate understanding of the invention, a number of terms and abbreviations as used herein are defined below as follows:

Trained: As used herein, the term “trained” refers to having been taught a particular skill or type of behavior through practice and instruction over a period of time.

Biological fluid: As used herein, the term “biological fluid” refers to a liquid that originates within the body of a mammal.

Biomarker: As used herein, the term “biomarker” refers to a measurable substance in an organism whose presence is indicative of some phenomenon such as disease, condition, infection, or environmental exposure. Biomarkers belong to five groups: cancer biomarkers, cardiac biomarkers, pathogenic biomarkers, molecular biomarkers, histologic biomarkers, and physiologic biomarkers. Examples of cancer biomarkers include bladder tumor-associated antigen (BTA), estrogen receptor, progesterone receptor, stem cell growth factor receptor (c-Kit), epidermal growth factor (EGFR), v-Ki-ras2 Kirsten rat sarcoma viral oncogene homologue (K-RAS), prostate-specific antigen (PSA), and Receptor tyrosine-protein kinase erbB-2 (HER2/NEU). Examples of cardiac biomarkers include troponin, myoglobin, creatine kinase, creatine kinase-MB, ischemia modified albumin, heart-type fatty acid-binding protein, natriuretic peptides, A-type atrial natriuretic peptide, B-type atrial natriuretic peptide (BNP), C-type atrial natriuretic peptide, N-terminal proBNP, C-reactive protein, soluble CD40 ligand, homocysteine, lipoprotein-associated phospholipase, myeloperoxidase, pregnancy-associated plasma protein A, choline, galectin 3, midregional proadrenomedullin, and copeptin. Examples of molecular biomarkers may include hemoglobin A1c, glucose, cholesterol, and high-density lipoproteins.

Neural Network: As used herein, the term “neural network” refers to a digital data structure with learnable weights, in which the computer's representation is learned rather than explicitly programmed. The details of learning algorithm and details of the data structure architecture may vary. Examples of a neural network may include a convolution neural network, recurrent neural network, transformer, or other neural network architecture, and the learning algorithm may entail one of supervised learning, unsupervised learning, or reinforcement learning.

### Systems and Methods for Predicting a Condition

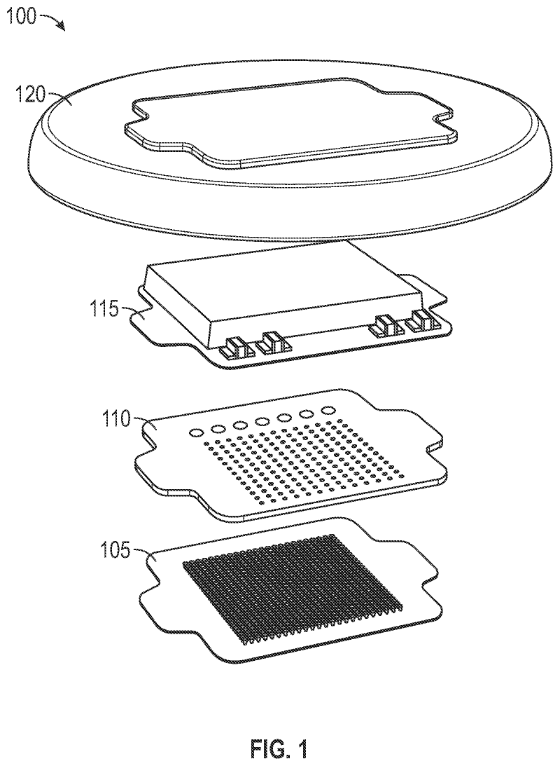

The present invention is directed to a system that is comprised of a patch 100, with the patch 100 comprising at least one hollow microneedle 105, an assay compartment 110, a microprocessor 115, and a cap 120, as shown in FIG. 1. In all embodiments, the microneedles 105 are hollow, having an open inner surface that allows biological fluid to enter the inner surface of the microneedles 105. In an embodiment, at least one hollow microneedle 105 may take the form of an array of microneedles. In another embodiment, at least one hollow nmicroneedle 105 may take the form of a single needle. Fluid collected in the assay compartment 110 may undergo various analyses. In an embodiment, the fluid may be subject to RNA analysis. In this instance, at least one of a nuclear run-on assay and ribosome profiling may be the assay type. In another embodiment, the fluid may be subject to DNA analysis. In this instance, at least one of DNAse footprinting assay, filter binding assay, and gel shift assay may be the assay types. In yet another embodiment, the fluid may be subject to protein analysis. In this instance, Bicinchoninic acid assay, Bradford protein assay, Lowry protein assay, enzyme-linked immunosorbent assay (ELISA), and secretion assay may be the assay types. In yet another embodiment, the fluid may be subject to cellular analysis. In this instance, at least one of polymerase chain reaction (PCR), microarray analysis, and flow cytometry analysis may be the techniques by which the fluid is analyzed. The system is capable of measuring a single biomarker, while it is versatile enough to measure multiple biomarkers. The fluid may take on more than one form. In an embodiment, the fluid may be whole blood. In another embodiment, the fluid may be plasma. In another embodiment, the fluid may be extracellular fluid. In yet another embodiment, the fluid may be interstitial fluid. In yet another embodiment, the fluid may be cerebrospinal fluid. In some embodiments, the length of the microneedle ranges from about 150 microns to about 1500 microns. In other embodiments, the length of the microneedle ranges from about 160 microns to about 1000 microns. In other embodiments, the length of the microneedle ranges from about 170 microns to about 750 microns. In other embodiments, the length of the microneedle ranges from about 180 microns to about 500 microns. In other embodiments, the length of the microneedle ranges from about 190 microns to about 225. In some embodiments, the width of the microneedle ranges from about 50 microns to about 250 microns. In other embodiments, the width of the microneedle ranges from about 60 microns to about 240 microns. In other embodiments, the width of the microneedle ranges from about 70 microns to about 230 microns. In other embodiments, the width of the microneedle ranges from about 80 microns to about 220 microns. In other embodiments, the width of the microneedle ranges from about 90 microns to about 210 microns. In other embodiments, the width of the microneedle ranges from about 100 microns to about 200 microns. In some embodiments, the diameter of the microneedle ranges from about 1 micron to about 25 microns. In other embodiments, the diameter of the microneedle ranges from about 2 microns to about 20 microns. In other embodiments, the diameter of the microneedle ranges from about 3 microns to about 15 microns. In other embodiments, the diameter of the microneedle ranges from about 4 microns to about 10 microns. The microneedle may be made from a variety of materials, such as silicon, titanium, aluminum oxide, zirconia, glass, nickel, iron, nitinol, stainless steel, and polymers such as PLA, PLGA, polycarbonate, and PMVE/MA copolymer. The microneedle has a hollow inner surface that allows biological fluids to enter the microneedle. The biomarker being analyzed may apply to various scenarios. Cancer biomarkers, cardiac biomarkers, pathogenic biomarkers, molecular biomarkers, histologic biomarkers, physiological biomarkers, and combinations thereof are analyzable. There may be several different types of assay compartments 110, depending on the type of biomarker being analyzed. Assay compartments 110 for cancer biomarkers, cardiac biomarkers, pathologic biomarkers, molecular biomarkers, histologic biomarkers, and physiologic biomarkers may be used with the patch 100. In some embodiments, it is possible to have assay compartments 110 that can analyze different types of biomarkers. Combinations of cancer biomarkers, cardiac biomarkers, pathologic biomarkers, molecular biomarkers, histologic biomarkers, and physiologic biomarkers may be used with the patch 100.

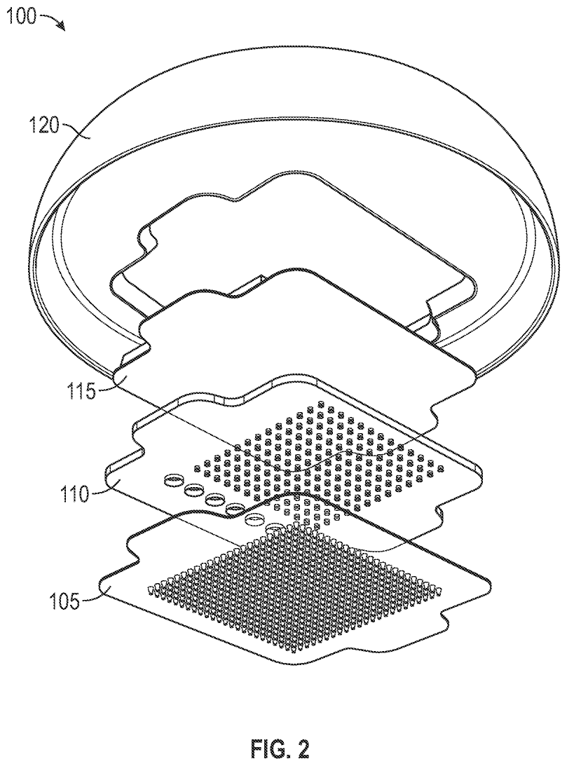

FIG. 2 is another depiction of the patch 100. The microprocessor 115 controls the rate of fluid collection by the microneedles 105. In embodiments, microneedles may be arranged on a base substrate in any suitable density. For example, a plurality of microneedles may be arranged in even or staggered rows in an array, wherein each microneedle is separated from its nearest neighboring microneedle by a distance between about 50% and about 200% of the height of the microneedle, (e.g., between about 75% and about 150% of the height of the microneedle, or by about equal to the height of the microneedle). Any suitable number of microneedles may be used. In one embodiment, a plurality of microneedles may include from about 5 microneedles to about 10,000 microneedles. In other embodiments, there may be from about 50 microneedles to about 1000 microneedles. In other embodiments, there may be from about 50 microneedles to about 200 microneedles. The patch 100 may be disposable in some embodiments. In other embodiments, the patch 100 is reusable. The microneedles 105 may have different cross-sections, such as circular, oval, triangular, square, rectangular, pentagonal, octagonal, and hexagonal.

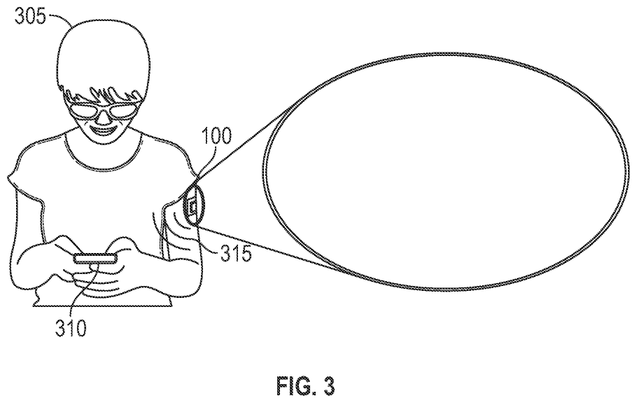

FIG. 3 shows a depiction of communication 315 between the patch 100 and the computing device 310 of a subject 305. The patch 100 communicates the detection of at least one biomarker, with the detection being indicative of the onset of a condition. Communication 315 acts as data being delivered from the patch 100 to the computing device 310, with the computing device 310 able to store the data. In some embodiments, the computing device 310 may be configured to be worn on a belt. In other embodiments, the computing device 310 may be carried in a pocket. In even other embodiments, the computing device 310 may be carried in a purse. In all embodiments, information is transmitted by the microprocessor 115 to the computing device 310 using, wireless communication 315 such as radio frequency (RF), near field communication, internet area network, personal area network, wireless LAN, LAN, body area network, or Bluetooth wireless. The receiver may maintain a continuous link with the sensor, or it may periodically receive information from the sensor. The microprocessor 115 and the computing device 310 may be synchronized using RFID technology or other unique identifiers. The computing device 310 may be provided with a display and user controls. The display may show, for instance, biomarker values, directional biomarker trend arrows and rates of change of biomarker concentration. The microprocessor 310 may also be configured with a speaker adapted to deliver an audible alarm or any auditory tone, such as high and low biomarker alarms. Additionally, the computing device 310 may include a memory device, such as a chip, that may store biomarker data for analysis by the subject or by a health care provider. Two way communication is possible in some embodiments, that is, the patch 100 may be in communication with a subject or a healthcare professional, and the healthcare professional may be in communication with the patch 100.

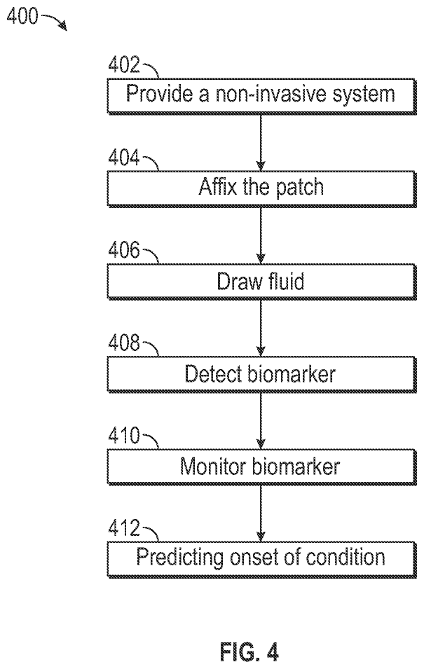

FIG. 4 is a flowchart 400 of the functioning of the system. Providing a system 402 comprises providing the patch 100, the patch comprising at least one hollow microneedle 105, an assay compartment 110, a microprocessor 115 (with the microprocessor further comprising circuitry that creates audio tones), and a cap 120. Optionally, the system may comprises a neural network that allows the collection of data from the patch 100, stored on a computing device 310, to be used to further train the system. Affixing the patch to a subject 404 allows microneedles 105 to make contact with the subject 305 to draw fluid 406 from the subject 305. Detecting a level of at least one biomarker 408, followed by the monitoring the level of the at least one biomarker 410, is needed to be able to predict a condition 412. Detecting 408 is done by the patch, with the data being sent to the computing device for monitoring 410. The more data that is collected, the more information the system can access to be able to form patient specific baselines for biomarkers and ultimately predict the onset of a condition 412, for example, in one embodiment, acute myocardial infarction.

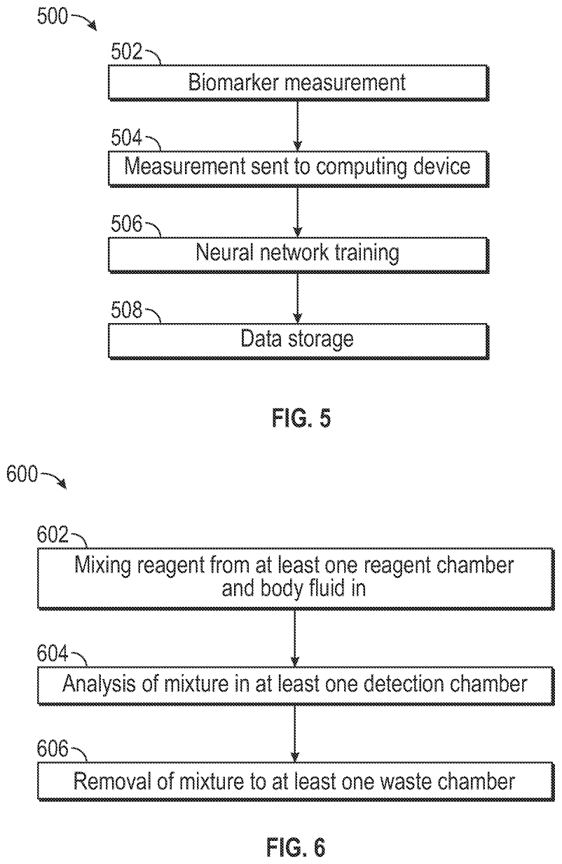

FIG. 5 is a flowchart of the training process 500. The patch making a measurement of biomarker 502 is proceeded by the measurement being sent to the computing device 504. At least one neural network trains on measurements as they are accumulated 506, better refining what normal baseline levels are and what is deemed an onset of a condition based on these measurements. The data is then stored in the computing device 508.

FIG. 6 depicts the assay compartment step 600. The body fluid (e.g. blood, interstitial fluid) and reagent from at least one reagent chamber are mixed 602 and analyzed in at least one detection chamber 604, after which the mixture is discarded in at least one waste chamber 606.

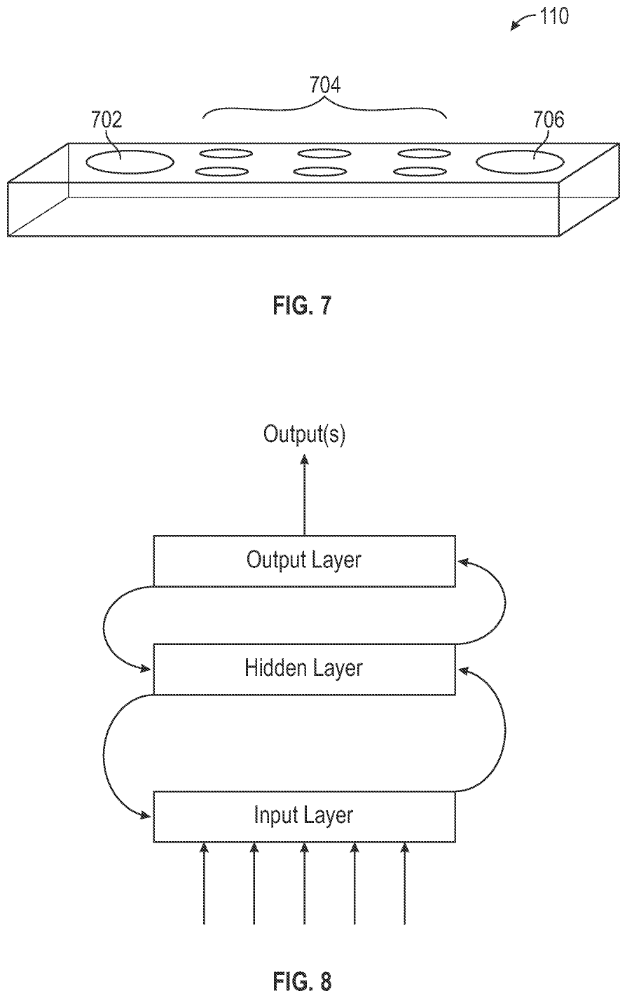

FIG. 7 shows an exemplary depiction of the assay compartment 110. The reagent chamber 702 holds reagent to mix with the fluid that is drawn from a subject of the system 100. The detection chamber 704 allows analysis of the biomarker or biomarkers of interest. The waste chamber 706 accepts waste product once analysis is complete. The assay compartment 110 may take on a variety of configurations. In one embodiment, the assay compartment 110 is a lab on a chip. In another embodiment, the assay compartment 110 is a biosensor. There are also several ways in which the assay compartment 110 may be in fluid communication with the microneedles 105. In an embodiment, tubing may be used to move biological fluid from the microneedles 105 to the assay compartment 110. In another embodiment, microfluidics or nanofluidics may be used to move biological fluid from the microneedles 105 to the assay compartment 110. A vacuum mechanism may be used to move biological fluid from the microneedles 105 to the assay compartment 110. Capillary action may also be used to move biological fluid from the microneedles 105 to the assay compartment 110. One of skill in the art would recognize that there are several other methods to move biological fluid from the microneedles 105 to the assay compartment 110 as long as the microneedles 105 are in close proximity to the assay compartment 110. The way in which it is determined that biomarker is present in a biological fluid varies. In an embodiment, a change in light absorbance of biological fluid in the detection chamber 704 of the assay compartment 110 indicates the presence of the biomarker(s) of interest. In another embodiment, a change in color of biological fluid in the detection chamber 704 of the assay compartment 110 indicates the presence of the biomarker(s) of interest. In other embodiments, there is a tripping sensor in communication with the detection chamber 704 of the assay compartment 110; this tripping sensor, when in contact with the biomarker(s) of interest in the biological sample, signals to the microprocessor 115 the presence of the biomarker(s) of interest.

FIG. 8 shows an exemplary depiction of information flow between the layers of a neural network. Each connection between nodes in the neural network has a weight associated with the node. Weights determine the effect that an input value has on the output value of the node. Before the network is trained, random values are selected for each of the weights. The weights change as the neural network is trained.

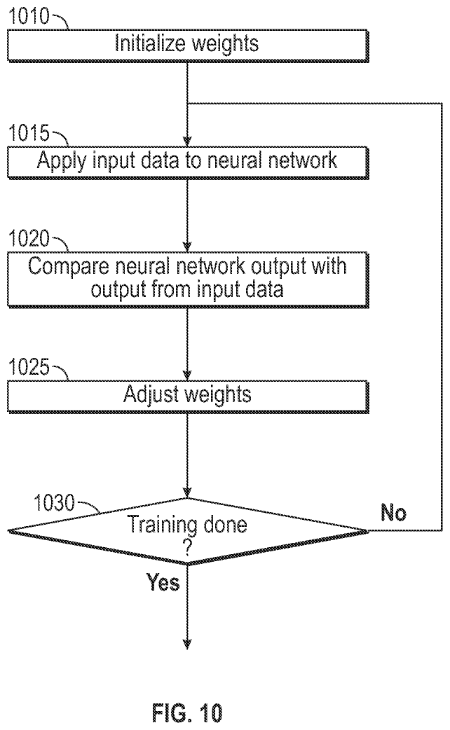

The weights are adjustable values that determine the prediction for a given set of input data; the neural network may adjust the weights automatically. Training the neural network means adjusting the weights in the neural network. Training a neural network requires that training data be assembled for use by the training means. The training means then implements the steps shown in FIG. 10. Weights are initialized to random values in step 1010. When retraining the neural network, step 1010 may be skipped so that training begins with the weights computed for the neural network from other training sessions. Input data is applied to the neural network in step 1015. This input causes the nodes in the input layer to generate outputs to the nodes of the hidden layer, which produces outputs to the nodes of the output layer, which produces the prediction; this process is commonly referred to as forward activation flow. Forward activation flow is depicted on the right side of FIG. 8.

Associated with the input data applied to the neural network in step 1015 is a wanted output value. In step 1020, the output produced by the neural network is compared with the desired output. The difference between the desired output and the output produced by the neural network is referred to as the error value. The error value is used to adjust the weights in the neural network, depicted in step 1025.

One suitable approach for adjusting weights is called back propagation. Back propagation is a training method in which an output error signal is sent back through the network, adjusting weights to minimize the error. The error between the neural network output value and the desired output from the input data is propagated back through the output layer and through the hidden layer or hidden layers. Back propagation distributes the overall error value to each of the nodes in the neural network, adjusting the weights associated with each node's inputs based on the error value allocated to it. This backward error flow is depicted on the left hand side of FIG. 8. As shown in step 1030, a test is used to determine whether training is complete. In an embodiment, the test may be a check that the error value falls below a threshold over a certain number of previous training iterations. In another embodiment, the test may be end training after a certain number of iterations. In yet another embodiment, a set of testing data is used for error measurement. The testing data may be generated so that it is mutually exclusive of the data used for training. If the error from using the testing data is below a certain amount, training is considered completed. If not, training continues.

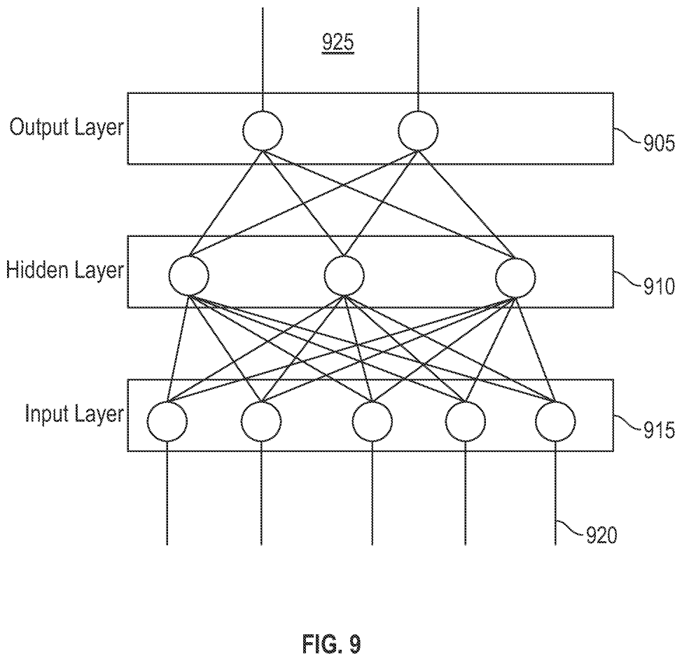

FIG. 9 depicts a neural network with an input layer, a hidden layer, and an output layer. a representative example of a neural network is shown. It should be noted that FIG. 9 is illustrative of one embodiment of a neural network; other embodiments of a neural network may be used. FIG. 9 shows an input layer 915, a hidden layer 910 and a output layer 905. The input layer 915 includes a layer of input nodes that take their input values 920 from the external input. The input data is used by the neural network to generate the output 925.

The hidden layer 910, while not required, is oftentimes used. It includes a set of nodes, with the outputs from nodes of the input layer 915 being used as inputs to each node in the hidden layer 910. The outputs of nodes of the hidden layer 910 are used as inputs to the nodes of the output layer 905. In an embodiment, there is one hidden layer 910. In other embodiments, there are more than one hidden layer 910.

In an embodiment, the output layer 905 may comprise one node. In other embodiments, the output layer 905 may comprise of more than one node. The input values of the output layer 905 come from the nodes of the hidden layer 910. The output of the nodes of the output layer 905 are the predictions 925 produced by the neural network using the input data 920.

Each connection between nodes in the neural network has a weight associated with it. Weights determine the effect an input value has on the output value of a node. Before the network is trained, random values are selected for each of the weights. The weights change as the neural network is trained.

In a neural network that has nodes having the same activation function, it is necessary to know the number of nodes in each layer. This determines the number of weights and ultimately total storage required to build the network. The more complex the network is, the more storage is needed.

The present invention contemplates other types of neural network configurations for use with a neural network. All that is required for a neural network is that the neural network be able to be trained and retrained to provide the needed predictions.

An exemplary embodiment of a feed forward neural network is shown in FIG. 9. Input data 920 is provided to input nodes in the input layer 915. The hidden layer 910 nodes each retrieve the input values from all of the inputs in the input layer 915. Each node has a weight associated with each input value. Each node is a product of an input value and its associated weight, and the sum of all these values is obtained. This sum is then used as an input to an activation function to produce an output for that node. In an embodiment, the processing for nodes in the hidden layer 910 may be performed in parallel. In another embodiment, the processing for nodes in the hidden layer 910 may be performed sequentially. In other embodiments, the processing for nodes in the hidden layer 910 may switch between being performed in parallel and sequentially. Each output is multiplied by its associated weight, and these values are summed. This sum is then used as input to produce the output data 925, with the output data 925 being a predicted value. An equivalent function can be achieved using analog means.

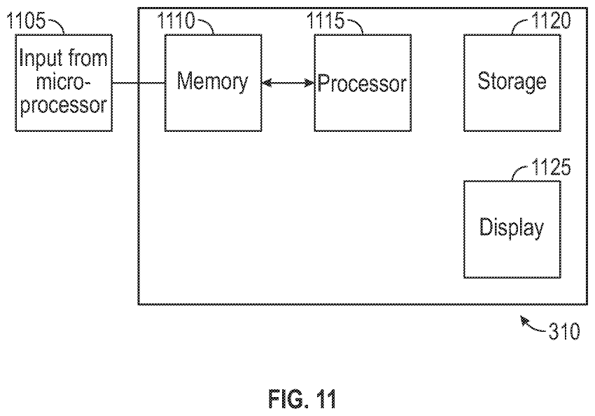

FIG. 11 depicts a block diagram of the interaction between the microprocessor 115 of the patch 100 and the computing device 310. The computing device 310 may include any decide that can accept data from the patch 100 for processing, such as a smartphone, computer, laptop, iPad, Apple watch, or other device. Any type of device capable of receiving one or more inputs and producing an output can be used as the computing device 310. Accordingly, the present embodiments should not be interpreted as limited to any type of hardware.

The computing device 310 includes a memory 1110 and a processor 1115. The memory 1110 provides the processor 1115 access to data and program information that is stored in the memory 1110 at execution time. Typically, the memory 1110 includes random access memory (RAM) circuits, read-only memory (ROM), flash memory, etc., or a combination of such devices. The processor 1115 may be, or may include, one or more programmable general-purpose or special-purpose microprocessors, digital signal processors (DSPs), programmable controllers, application specific integrated circuits (ASICs), programmable logic devices (PLDs), etc., or a combination of such hardware-based devices. The memory 1110 receives input from the microprocessor 1105, this input originating in the microprocessor of the patch 100. The processor 1115 executes on the input 1105 relayed to the memory 1110. Collection of the input 1105 informs the training of the neural network, with new data being compared with already stored data and neural network output being compared with output from input data. Input 1105 from the patch 100 may include information on time to peak of biomarker levels, biomarker concentration, biomarker levels, biomarker presence, changes in biomarker concentration, changes in biomarker levels, and changes in biomarker presence. Collection of input 1105 and historical data may be used to determine a threshold or a score, after which an action is suggested or deemed required. After exceeding a threshold, such action may be go to a healthcare facility (e.g. hospital), visit a healthcare professional (i.e. physician, physician's assistant), and below the threshold, the action may be no action required, or self monitor. The collection of data from the patch 100 may be used to determine the thresholds or score, with such thresholds and scores being used to make predictions on the onset of diseases and conditions, including cancer, diabetes, coronavirus disease, acute myocardial infarction, infectious diseases, biological poisons, radiation, tropical diseases, and COVID-19.

A software and/or firmware component may be stored in storage 1120 available to the computing device 310, and loaded into the memory 1110 at execution time. The storage 1120 may be any non-transitory computer readable media including, but not limited to, a hard disk, EEPROM (electrically erasable programmable read only memory), a memory stick, or any other storage device type. The memory 1110 may receive an input and store the input as an input parameter for later processing. Storage 1120 may be local or remote.

In certain embodiments, there may be downloadable software that a user may download from a remote server through a wired or wireless connection. For example, the subject may access the server using an application already installed on the subject's computing device 310. The subject may then download and install an application for assistance with displaying input 1105 from the patch 100. The application may be configured to suit the subject's personal preferences and/or settings. Such a configuration may be done manually, such as by selecting various options from menus, or automatically. In automatic configuration, the subject's preferences and/or settings are taken from information stored on the computing device 310. An embodiment of manually configuring the subject's personal preferences and/or settings may include a graphical subject interface (GUI). The GUI may be displayed on a display 1125 of the computing device 310 when the subject inputs a command to configure personal preferences and/or settings. Input 1105 from the patch 100 may be displayed on the display 1125. Output from the neural network may be displayed on the display 1125. Historical data may be displayed on the display 1125. Biomarker concentrations, biomarker levels, presence of biomarker, changes in biomarker concentrations, changes in biomarker levels, and changes in presence of biomarkers may be displayed on the display 1125.

The systems and methods disclosed herein may be implemented via one or more components, systems, servers, appliances, other subcomponents, or distributed between such elements. When implemented as a system, such systems may include an/or involve, inter alia, components such as software modules, general-purpose CPU, RAM, etc., found in general-purpose computers. In implementations where the innovations reside on a server, such a server may include or involve components such as CPU, RAM, etc., such as those found in general-purpose computers.

Additionally, the systems and methods herein may be achieved via implementations with disparate or entirely different software, hardware and/or firmware components, beyond that set forth above. With regard to such other components (e.g., software, processing components, etc.) and/or computer-readable media associated with or embodying the present implementations, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the systems and methods may be achieved via or performed by logic and/or logic instructions including program modules, executed in association with such components or circuitry, for example. In general, program modules may include routines, programs, objects, components, data structures, etc., that perform particular tasks or implement particular instructions herein. The embodiments may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, where media of any type herein does not include transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the implementations described herein or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the implementations herein, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

Moreover, the above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So for example performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y and Z to obtain the benefit of such steps. Thus method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It should further be appreciated that the methods above are provided by way of example. Absent an explicit indication to the contrary, the disclosed steps may be modified, supplemented, omitted, and/or re-ordered without departing from the scope of this disclosure.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

### EXAMPLES

Aspects of the present teachings may be further understood in light of the following examples, which should not be construed as limiting the scope of the present teachings in any way.

### Example 1—Biomarker Analysis

A patch is affixed to a subject, preferably the skin. Microneedles puncture the skin to have access to the subject's biological fluid, such as the blood. A pumping mechanism moves blood from the body through the microneedles. The blood enters an assay compartment, designed to prevent backflow of the blood back into the subject. A microprocessor controls the rate of biological fluid collection (i.e. controls the pumping mechanism) and sends assay data (information regarding the biomarker) wirelessly to a computing device, a smartphone for instance. Circuitry sends out an audio tone to convey the microprocessor's completion of analyzing assay data. The smartphone stores the assay data.

### Example 2—Biomarker Analysis with Prediction of an Onset Condition

As in example 1, the patch transfers assay data to the computing device, with the data being collected. Once a multitude of data is collected, at least one neural network in the computing device may receive assay data and be trained to make predictions on the onset of a condition based on a threshold biomarker measurement (which may include the use of multiple biomarkers simultaneously). Less than the threshold indicates a lesser likelihood of the onset of a condition. Above a threshold indicates a greater likelihood of the onset of a condition. As more and more data sets are collected, the threshold is better dialed in. Data sets for children, adult females, and adult males, are possible, as well as thresholds based on ethnicity, weight, and other factors.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

What is claimed:

1. A system for detecting a condition in a subject, the system comprising:
a patch further comprising:
at least one hollow microneedle;
an assay compartment in fluid communication with the at least one hollow microneedle;
a microprocessor that controls collection rate of a fluid from the subject via the at least one hollow microneedle;
wherein the microprocessor is in electronic communication with a computing device that displays presence of the condition.

2. The system of claim 1, wherein the at least one hollow microneedle draws fluid from the subject to monitor the at least one biomarker by way of at least one of an osmotic gradient, a pumping mechanism, vacuum, capillary action, surface tension, microfluidics, nanofluidics, and tubing.

3. The system of claim 2, wherein the at least one hollow microneedle draws fluid from a subject at least one of continuously and intermittently.

4. The system of claim 3, wherein data associated with the fluid is transferred from the microprocessor to the computing device.

5. The system of claim 4, wherein at least one factor is used to predict an onset of the condition, with the at least one factor being time to peak of biomarker levels.

6. The system of claim 5, wherein knowledge of the at least one factor is used to recommend a course of action.

7. The system of claim 4, wherein at least one neural network accepts the data, and a collection of output data of the at least one neural network is used to predict the condition.

8. The system of claim 2, wherein the at least one hollow microneedle gains access to the fluid by at least one of subcutaneously, percutaneously, dermally, sub-dermally, intra-peritoneally, and peritoneally.

9. The system of claim 2, wherein the at least one biomarker is detected at least one of before the condition, during the condition, and after the condition.

10. A method of predicting a condition in a subject, the method comprising:
providing a system for detecting a condition in a subject, the system comprising
a patch further comprising:
at least one hollow microneedle;
an assay compartment in fluid communication with the at least one hollow microneedle;
a microprocessor that controls collection rate of a fluid from the subject via the at least one hollow microneedle;
wherein the microprocessor is in electronic communication with a computing device that displays presence of the condition.
affixing the patch to the subject so that the at least one hollow microneedle accesses a fluid;
drawing the fluid through the at least one hollow microneedle;
detecting presence of at least one biomarker;
monitoring the at least one biomarker; and
predicting an onset of a condition based on the at least one biomarker.

11. The method of claim 10, wherein the audio tone serves to indicate at least one of an initiation of an assay, a completion of an assay, results of the assay being sent to the computing device, and a warning for the presence of a condition.

12. The method of claim 10, wherein at least one hollow microneedle draws fluid from the subject to detect the presence of at least one biomarker by way of at least one of an osmotic gradient, a pumping mechanism, vacuum, capillary action, surface tension, microfluidics, nanofluidics, and tubing.

13. The method of claim 12, wherein the at least one hollow microneedle draws fluid from the subject at least one of continuously and intermittently.

14. The method of claim 13, wherein data associated with the fluid is transferred from the microprocessor to the computing device.

15. The method of claim 14, wherein at least one factor is used to predict an onset of the condition, with the at least one factor being time to peak of biomarker levels.

16. The method of claim 15, wherein knowledge of the at least one factor is used to recommend a course of action.

17. The method of claim 14, wherein at least one neural network accepts the data, and a collection of output data of the at least one neural network is used to predict the condition.

18. The method of claim 12, wherein the at least one hollow microneedle gains access to the fluid by at least one of percutaneously, dermally, sub-dermally, intra-peritoneally, and peritoneally.

19. The method of claim 12, wherein the at least one biomarker is detected at least one of before the condition, during the condition, and after the condition.

20. The method of claim 12, wherein a threshold associated with the at least one biomarker determines whether treatment is necessary.

21. The system of claim 1, wherein the assay compartment prevents backflow into the subject.

22. The system of claim 21, wherein the assay compartment comprises at least one reagent chamber, at least one detection chamber, and at least one waste chamber.

23. The system of claim 1, wherein an adhesive affixes the patch to the subject.

24. The system of claim 1, further comprising circuitry that creates an audio tone based on assay data and a cap that ensures integrity of the system; and
optionally at least one neural network that trains the system to better predict the condition, with the condition being at least one of acute myocardial infarction, infectious diseases, biological poisons, radiation, tropical diseases, and COVID-19.

25. A kit comprising at least one patch, the at least one patch comprising
at least one hollow microneedle;
an assay compartment in fluid communication with the at least one hollow microneedle;
a microprocessor that controls collection rate of a fluid from the subject via the at least one hollow microneedle;
wherein the microprocessor is in electronic communication with a computing device that displays presence of a condition; and
instructions for use.

26. A system for detecting a condition in a subject, the system comprising
a patch further comprising:
at least one hollow microneedle;
an assay compartment in fluid communication with the at least one hollow microneedle;
a microprocessor that controls collection rate of a fluid from the subject via the at least one hollow microneedle;
wherein the microprocessor is in electronic communication with a computing device that displays presence of the condition;
instructions for use;
circuitry that creates an audio tone based on assay data and a cap that ensures integrity of the system; and
optionally at least one neural network that trains the system to better predict the condition, with the condition being at least one of acute myocardial infarction, infectious diseases, biological poisons, radiation, tropical diseases, and COVID-19;
wherein the at least one hollow microneedle draws fluid from the subject to monitor presence of at least one biomarker by way of at least one of an osmotic gradient, a pumping mechanism, vacuum, capillary action, surface tension, microfluidics, nanofluidics, and tubing; the at least one hollow microneedle draws fluid from the subject at least one of continuously and intermittently; data associated with the fluid is transferred from the microprocessor to the computing device; at least one factor is used to predict an onset of the condition, with the at least one factor being time to peak of biomarker levels; knowledge of the at least one factor is used to recommend a course of action; the at least one neural network accepts the data, and a collection of output data of the at least one neural network is used to predict the condition; the at least one hollow microneedle gains access to the fluid by at least one of subcutaneously, percutaneously, dermally, sub-dermally, intra-peritoneally, and peritoneally; the at least one biomarker is detected at least one of before the condition, during the condition, and after the condition; the assay compartment prevents backflow into the subject; the assay compartment comprises at least one reagent chamber, at least one detection chamber and at least one waste chamber; and an adhesive affixes the patch to the subject.
